(19) United States
(12) Patent Application Publication
(10) Pub. No.: US 2014/0171714 A1
(43) Pub. Date: Jun. 19, 2014

(54) PROCESS TO INCREASE HYDROGEN PRODUCTION WITHOUT LOSS OF STEAM PRODUCTION IN A STEAM METHANE REFORMER

(71) Applicant: L'Air Liquide Societe Anonyme Pour I'Etude et I'Exploitation des Procedes Georges Claude, (US)

(72) Inventors: Pascal Tromeur, Houston, TX (US); Bhadra S. Grover, Sugar Land, TX (US); Tony Thampan, Bear, DE (US)

(73) Assignee: L'Air Liquide Societe Anonyme Pour I'Etude et I'Exploitation des Procedes Georges Claude, Paris (FR)

(21) Appl. No.: 13/717,821

(22) Filed: Dec. 18, 2012

(51) Int. Cl. C07C 4/02

(52) U.S. Cl. CPC C07C 4/025
USPC 585/752; 422/649

(57) ABSTRACT

A process for pre-reforming a hydrocarbon containing stream prior to admission into a steam methane reformer is provided. This process includes a system with two pre-reformer reactors in series, wherein an oxygen stream is combined with the partially reformed outlet stream of the first pre-reformer reactor, then the combined stream is introduced into the second pre-reformer reactor.

### BACKGROUND

To increase Hydrogen production for an steam methane reformer (SMR) plant, there are different options available depending upon the plant design. In the case where the reformer capacity becomes limiting, additional hydrogen must be produced in a different unit operation. Although hydrogen can be produced by an additional reformer this is a very costly option. A preferable alternative has typically been the addition of a pre-reformer upstream of the reformer.

The conventional adiabatic pre-reformer offers the following benefits. Hydrogen production is typically increased by between 8 and 10%. The pre-reformer provides the ability to process heavier hydrocarbon feeds. Such heavy hydrocarbon feed would tend to coke in a main reformer. The pre-reformer provides the ability to lower the steam export, as heat is recovered for the pre-reforming process.

However, the limitation of the conventional pre-reformer is the inlet temperature. Typically, a design maximum inlet temperature of less than 500 C. (932 F.) is required, as higher temperatures can lead to coking, which results in low performance. To overcome this issue the following are options. The addition of a second pre-reformer which operates at a much higher temperature. Although this increases the temperature limit, there is corresponding decrease in the duty available for steam production and subsequently a steam export reduction.

Also, it is possible to use an oxygen blown secondary reactor with the primary SMR. It is sometimes suggested to utilize an oxygen fired reactor to debottleneck plants.

In the case where additional hydrogen is required, oxygen may be fed into the reactor to increase the hydrogen production. However the limitations are the following. A reactor that is designed for oxy-combustion may be costly. The use of an oxygen flame's high temperatures with a heavy hydrocarbon stream can result in coking/soot formation leading to reactor performance losses. The injection of oxygen directly into the feed can lead to gas flammability issues.

Hence, a need exists in the industry, for a means for safely increasing hydrogen production in an SMR, without the loss of steam production.

### SUMMARY

A process for pre-reforming a hydrocarbon containing stream prior to admission into a steam methane reformer is provided. This process includes a system with two pre-reformer reactors in series, wherein an oxygen stream is combined with the partially reformed outlet stream of the first pre-reformer reactor, then the combined stream is introduced into the second pre-reformer reactor.

### BRIEF DESCRIPTION OF THE FIGURES

FIG. 1 illustrates a typical state-of-the-art steam methane reforming plant, in accordance with existing art.

FIG. 2 illustrates a typical state-of-the-art steam methane reforming plant, in accordance with existing art.

FIG. 3 illustrates a typical state-of-the-art steam methane reforming plant, in accordance with existing art.

FIG. 4 illustrates one embodiment of the present invention.

FIG. 5 illustrates another embodiment of the present invention.

### DESCRIPTION OF PREFERRED EMBODIMENTS

Illustrative embodiments of the invention are described below. While the invention is susceptible to various modifications and alternative forms, specific embodiments thereof have been shown by way of example in the drawings and are herein described in detail. It should be understood, however, that the description herein of specific embodiments is not intended to limit the invention to the particular forms disclosed, but on the contrary, the intention is to cover all modifications, equivalents, and alternatives falling within the spirit and scope of the invention as defined by the appended claims.

It will of course be appreciated that in the development of any such actual embodiment, numerous implementation-specific decisions must be made to achieve the developer's specific goals, such as compliance with system-related and business-related constraints, which will vary from one implementation to another. Moreover, it will be appreciated that such a development effort might be complex and time-consuming, but would nevertheless be a routine undertaking for those of ordinary skill in the art having the benefit of this disclosure.

The present process solves the problem of increasing hydrogen production at an existing plant in a safe and economic manner. The use of an oxygen stream injected after the initial pre-reformer (1˜2% mole fraction which is below the flammability limits) results in a significant increase in hydrogen production (4˜12%) while avoiding any major redesign of the plant except the installation of an additional pre-reformer stage.

The present process allows the hydrogen production increase without export steam reduction, by utilizing oxygen enrichment. A secondary reactor is installed downstream of the conventional adiabatic reactor, and oxygen is introduced before this reactor. The present process may be considered counterintuitive because oxygen injection downstream of the initial pre-reformer is typically avoided since hydrocarbon conversion has already occurred, and the additional oxygen required for additional conversion will result in significant redesign to the plant.

However the following unique and unexpected aspects have been discovered. Due to the endothermic nature of reforming, a large temperature drop occurs in the initial adiabatic pre-reformer reactor, resulting in a lower exit temperature. Oxygen introduced after the initial reactor, but prior the secondary reactor results in a pre-reformer stage that can be isothermal, thereby resulting in higher hydrogen production versus the case without the oxygen stage.

The exit temperature of the initial reactor is sufficiently low to allow for the introduction of an oxygen stream while remaining below the flammability limit, thereby allowing the injection of oxygen directly into the stream to provide flameless combustion/catalytic combustion in the second reactor. Other injection zones—such as before the initial pre-reformer and prior the SMR do not allow significant oxygen injection into the feed while remaining below the flammability zones. Surprisingly, the injection of 1˜3% oxygen in this location, provides a 4˜12% hydrogen increase. Heavy hydrocarbons (i.e. C2+) can essentially be eliminated in a stream exiting the first reactor, allowing operation at higher temperatures (and thus use of oxygen) in downstream units

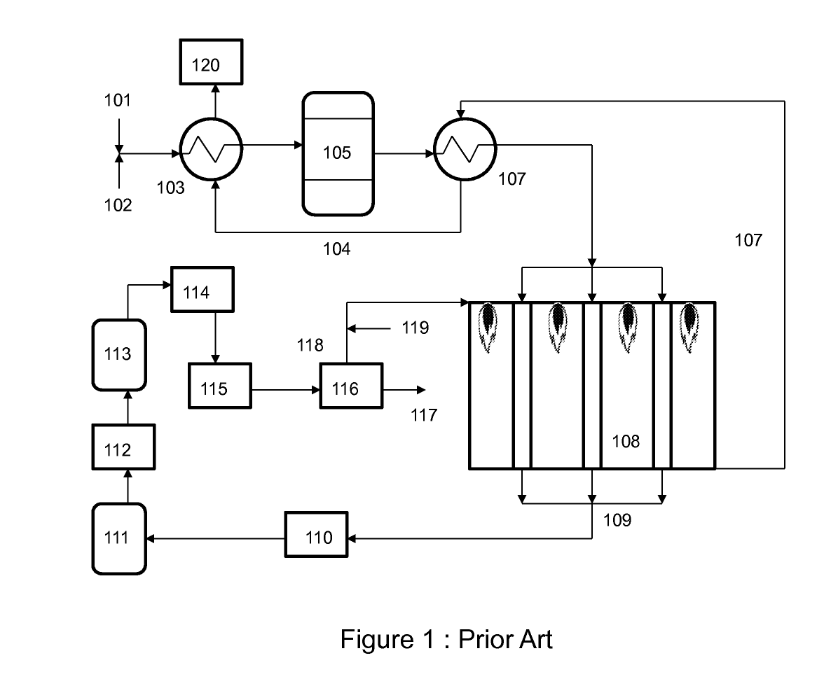

A typical state-of-the-art steam methane reforming plant, with a pre-reformer, is illustrated in FIG. 1. The feed is purified to remove sulfur and other contaminants 101, mixed with steam 102 and preheated 103 with a flue gas 104 before fed to the pre-reformer 105. Additional heat may be removed from the flue gas in downstream boilers 120. As reforming is an endothermic process, the gas is cooler at the pre-reformer outlet and must be preheated 106 with flue gas 107 again, before being fed to the SMR 108. The syngas leaving the reformer 109 is cooled to produce steam (boiler 110), processed in the high temperature shift reactor 111 for the water gas shift reaction followed by cooling by feed preheating and steam production 112. The syngas is further processed in a low temperature shift 113 to convert remaining carbon monoxide to hydrogen. To remove carbon dioxide (if desired) and H₂, the syngas must be cooled and water removed, which is done in heat exchangers and condensate removal vessels 114, before removal of carbon dioxide 115. Finally the gas is sent to PSA 116 for separation of the hydrogen from the other constituents. The hydrogen is exported 117 for use while the remaining constituents are utilized as burner fuel 118. Burner fuel 118 may be combined with additional natural gas 119, as needed. Simulations are shown for the base case (Case 1) are shown in Table 1.

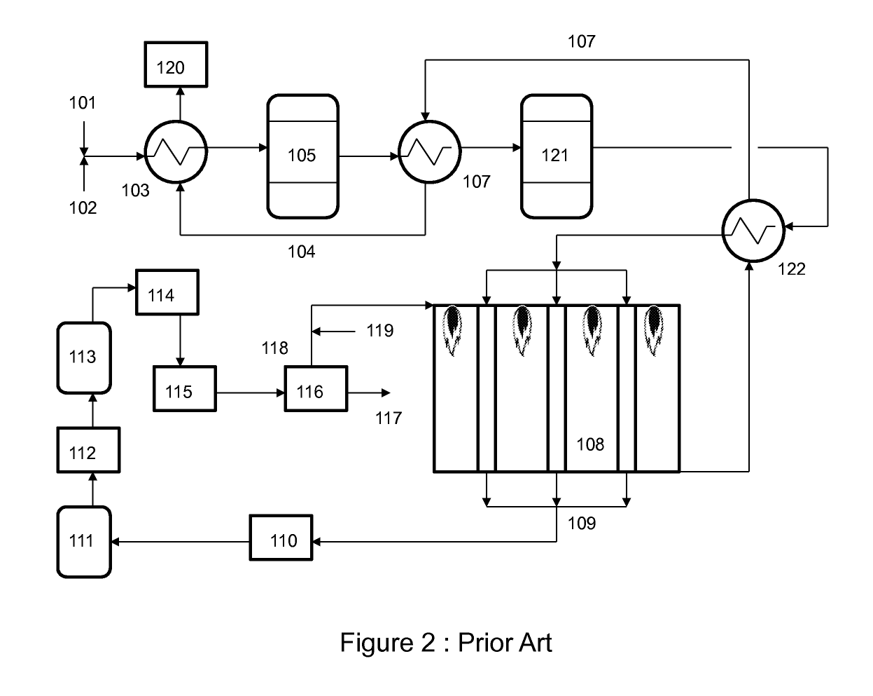

To further increase production, an additional pre-reforming reactor 121 and heat exchanger 122 have been added, as shown in FIG. 2. In order to avoid confusion, the same element numbers are used in FIG. 2, as previously detailed in FIG. 1. However this scheme involves substantial retrofit of the flue gas duct and results in a steam production reduction. As shown in Table 1, the resulting H₂ production increases by 6% but the steam is reduced by 43% (Case 2).

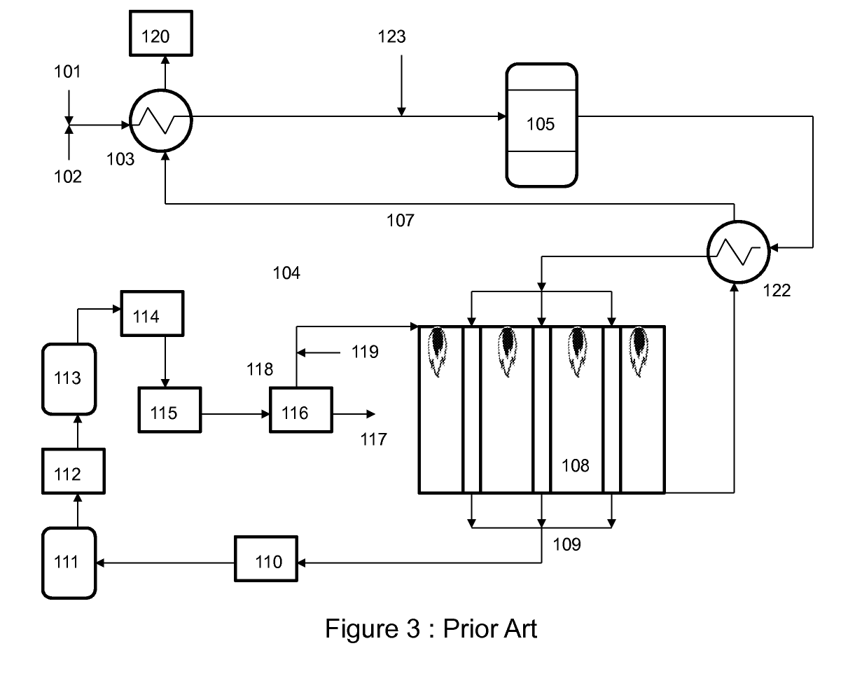

FIG. 3 shows the process layout with oxygen 123 injected before the pre-reformer 105. In order to avoid confusion, the same element numbers are used in FIG. 3, as previously detailed in FIGS. 1 and 2. In another embodiment, pre-reformer reactors may be operated in parallel The major limitation in this case is that if combustion is to be avoided, the oxygen injection must be below the flammability limits (−450° C./842° F.). However lowering the feed temperature to the pre-reformer results in an expensive plant redesign which can also result in excess steam production. The use of a oxygen combustion reactor with an oxygen burner in the reactor involves additional cost If any higher hydrocarbons (C2+) are present in the stream, the temperature (and thus oxygen) must be limited to avoid coke formation. For example with propane, evidence of coking has been reported at 560° C./1040° F.

Case 3 in Table 1 shows that oxygen injection at levels to remain below 1040° F. (to avoid coking), the H2 production is increased by 6%, while the steam increases by

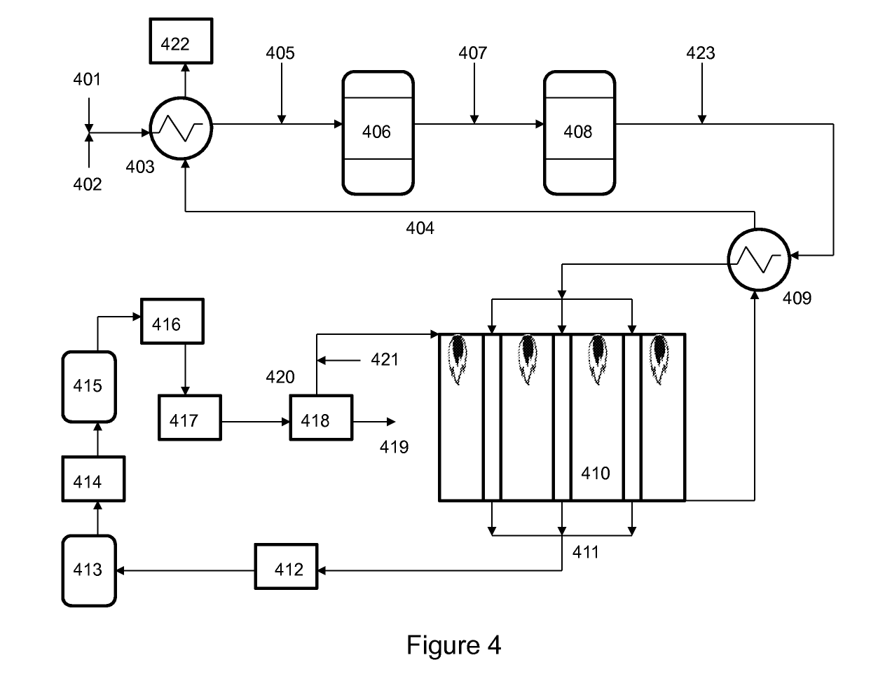

FIG. 4 shows the current inventive process. This process introduces the injection of oxygen between the two pre-reformers. Thermodynamic simulations have shown that there is essential no C1+ hydrocarbons (<500 ppm) remaining in the feed after the first pre-reformer. Thus oxygen may be introduced safely and efficiently at the location indicated by stream 407. As the results in Table 1 indicate, by injecting oxygen in the manner described in FIG. 4 and Table 1, a 14% increase in hydrogen and a 44% increase in steam production may be observed.

The feed is purified to remove sulfur and other contaminants 401, mixed with steam 402 and preheated 403 with a flue gas 404 before fed to first pre-reformer 406. Additional heat may be removed from the flue gas in downstream boilers 422. The heated feed and steam stream is then combined with first oxygen stream 405 and introduced into first pre-reformer 406. The partially reformed stream exiting first pre-reformer 406 is then combined with second oxygen stream 407 and introduced into second pre-reformer 408. After exchanging heat with flue gas 409, this pre-reformed stream is then fed into SMR 410. Third oxygen stream 423 may be introduced into the pre-reformed stream exiting second pre-reformer 408, prior to admission into SMR 410.

The syngas leaving the reformer 410 is cooled to produce steam (boiler 412), processed in the high temperature shift reactor 413 for the water gas shift reaction followed by cooling by feed preheating and steam production 414. The syngas is further processed in a low temperature shift 415 to convert remaining carbon monoxide to hydrogen. To remove carbon dioxide (if desired) and hydrogen, the syngas must be cooled and water removed, which is done in heat exchangers and condensate removal vessels 416, before removal of carbon dioxide 417. Finally the gas is sent to PSA 418 for separation of the hydrogen from the other constituents.

The hydrogen is exported 419 for use while the remaining constituents are utilized as burner fuel 420. Burner fuel 420 may be combined with additional natural gas 421 as needed.

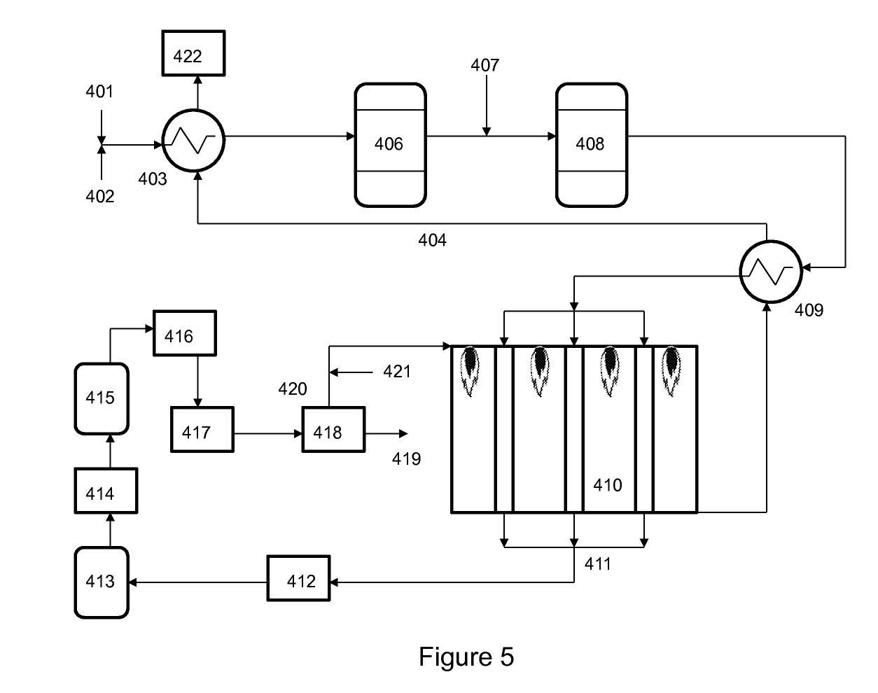

Referring to the case as shown in FIG. 5, the oxygen is injected at location 2. In order to avoid confusion, the same element numbers are used in FIG. 4, as previously detailed in FIG. 4. This embodiment is based on the temperature at the location indicated by stream 407, to enable flameless combustion in second pre-reformer reactor 408. The flammability zone is dependent upon the auto ignition temperature and is estimated as follows

### Flammability Data Description

Using the GRI mechanism to simulate the reaction for a specific rich methane mixture, the auto ignition temperature can be calculated. The detailed chemical mechanism used is the GRI-Mech Version 3.0 7/30/99 CHEMKIN-II. With a mixture composition of 22% methane/2.5% O2/H2O (equivalent ratio=17.6), the minimum AIT calculated is 842 F. (at 33 bar). In general the temperature prediction is 4˜5% lower than experimental measurements.

Considering that the temperature entering the pre-reformer 1 is 940 F. (temperature margin chosen to avoid coking),the exiting temperature is at 847 F. Thus it can be observed the only location that allows oxygen injection, while remaining below the flammability zone is after the initial pre-reformer. Thus the outlet temperature of second pre-reformer 408 is low enough to allow injection of 1˜2% oxygen while remaining below the flammability limit which allows injection of the oxygen directly into the feed.

### Case 5A

Case 5A is the case where the 2% v/v oxygen is injected in to the feed at the location indicated by stream 407, where the conditions are such that combustion is not expected to occur. When the feed enters the reactor, the catalyst allows oxidation and the oxidation provides the energy required for reforming. Case 5A in Table 1 shows the benefits of a hydrogen increase of 4% and a steam increase of 18%. In the case where the hydrogen is required, but no additional steam, it is possible to increase hydrogen production with minimum retrofit. (Case 5b). Case 5b shows that it is possible with oxygen injection to increase the preheat on an existing feed pre-heater by 52 F. resulting in an hydrogen increase of 7% with only a 3% steam increase.

TABLE 1
Results obtained with simulations

| | TITLE | | | | | |
|---|---|---|---|---|---|---|
| | CASE 1 | CASE 2 | CASE 3 | CASE 4 | CASE 5A | CASE 5B |
| | Description | | | | | |
| | | | | | | SMR w/O2 inject. at |
| | | | SMR w/O2 | | SMR w/O2 inject. at | Prereformer #2 - at |
| | SMR w/1 | SMR w/2 | inject. at | SMR w/O2 inject. at | Prereformer #2 - at | flammability limits |
| | Prereformer | Prereformer | Prereformer #1 | Prereformer #1 & #2 | flammability limits | (Higher Temp Feed) |
| H2 Production | 100 | 106 | 106 | 114 | 104 | 107 |
| (MMSCFD) | | | | | | |
| Steam | 76 | 43 | 96 | 110 | 90 | 78 |
| Production | | | | | | |
| (MMBtu/hr) | | | | | | |
| NG Feed | 1294 | 1373 | 1399 | 1541 | 1367 | 1405 |
| (MMBtu/hr) | | | | | | |
| NG Fuel | 203 | 180 | 208 | 203 | 208 | 197 |
| (MMBu/hr) | | | | | | |
| NG Consumption | 1497 | 1553 | 1607 | 1744 | 1575 | 1602 |
| (MMBtu/hr) | | | | | | |
| O2 Consumption | 0 | 0 | 10 | 11 (injected at | 7 | 8 |
| (Mlbs/hr) | | | | prereforemer 1) + 10 | | |
| | | | | (injected at | | |
| | | | | prereformer 2) | | |
| Prereformer #1 | 847 | 847 | 1047-1061 | 1047-1062 | 847 | 847 |
| Outlet Temp (F.) | | | | | | |
| Prereformer #2 | na | 1200 | na | 1062 | 847 | 847 |
| Inlet Temp (F.) | | | | | | |
| Prereformer #2 | na | 1041 | na | 1197 | 1013 | 1013 |
| Outlet Temp (F.) | | | | | | |
| Reformer Feed | 1200 | 1200 | 1200 | 1200 | 1200 | 1252 |
| Inlet Temp (F.) | | | | | | |
| Plant Efficiency | 0.811 | 0.806 | 0.810 | 0.809 | 0.810 | 0.809 |
| H2 Efficiency | 0.761 | 0.779 | 0.751 | 0.746 | 0.753 | 0.761 |

The simulations were done with the following constant:

| | |
|---|---|
| Prereformer #1 Inlet | 940 |
| Temp (F.) | |
| S/C to Reformers | 2.9 |
| S/C to Prereformers | 1.8 |
| Reformer Syngas | 1652 |
| Temp out (F.) | |
| Reformer BWT (F.) | 1926 |
| Reformer Adsorbed | 360 |
| Duty (MMBtu/hr) | |

What is claimed is:

1. A process for pre-reforming a hydrocarbon containing stream prior to admission into a steam methane reformer, comprising two pre-reformer reactors in series, wherein an oxygen stream is combined with the partially reformed outlet stream of the first pre-reformer reactor, then the combined stream is introduced into the second pre-reformer reactor.

2. The process of claim 1, wherein the partially reformed outlet stream of the first pre-reformer reactor has less than 500 ppm of C2+ hydrocarbons.

3. The process of claim 1, wherein the partially reformed outlet stream of the first pre-reformer reactor has a temperature above the auto ignition temperature.

4. The process of claim 3, wherein flameless, catalytic combustion takes place in the second pre-reformer reactor.

5. The process of claim 3, wherein the temperature of the partially reformed outlet stream of the first pre-reformer reactor is greater than 840 F.

6. The process of claim 5, wherein the temperature of the partially reformed outlet stream of the first pre-reformer reactor is greater than 850 F.

7. The process of claim 6, wherein the temperature of the partially reformed outlet stream of the first pre-reformer reactor is greater than 860 F.

8. The process of claim 1, further comprising combining a second oxygen stream with the feed stream for the first pre-reformer reactor.

9. The process of claim 1, further comprising combining a third oxygen stream with the outlet stream from the second pre-reformer reactor.

10. An apparatus for pre-reforming a hydrocarbon containing stream prior to admission into a steam methane reformer, comprising two pre-reformer reactors in series, an oxygen admission means for combining an oxygen stream with the partially reformed outlet stream of the first pre-reformer reactor, and a means for introducing the combined stream into the second pre-reformer reactor.

11. The apparatus of claim 10, further comprising a second oxygen admission means for combining a second oxygen stream with the feed stream for the first pre-reformer reactor, and a means for introducing the second combined stream into the first pre-reformer reactor.

12. The apparatus of claim 10, further comprising a third oxygen admission means for combining a third oxygen stream with the outlet stream from the second pre-reformer reactor, and a means for introducing the third combined stream into the steam methane reformer.
